(19) United States
(12) Patent Application Publication
(10) Pub. No.: US 2014/0220614 A1
(43) Pub. Date: Aug. 7, 2014

(54) NON-INVASIVE PRENATAL GENDER TESTING

(71) Applicant: Organic Gender, LLC, Tampa, FL (US)

(72) Inventor: Joe Ben Sirmans, Tampa, FL (US)

(73) Assignee: Organic Gender, LLC, Tampa, FL (US)

(21) Appl. No.: 14/173,460

(22) Filed: Feb. 5, 2014

Related U.S. Application Data
(60) Provisional application No. 61/760,873, filed on Feb. 5, 2013.

(51) Int. Cl. G01N 33/74

(52) U.S. Cl. CPC G01N 33/74
USPC 435/29

(57) ABSTRACT

Prenatal gender testing apparatus, system and method for a fetus. The gender testing utilizes organic wheat seeds and organic barley seeds in a nutrient-rich soil, all contained within clay pots. A prospective mother supplies each soil-seed mixture with her own urine (diluted with water at an approximate 1:1 ratio) over the course of several days with a plurality of applications of said diluted urine. As the seeds begin to sprout, the strength, speed, and height of growth determines whether the fetus is male or female. Whichever seed (wheat or barley) results in the higher strength, speed, and height determines gender of the fetus. Thus, if the wheat seeds have the greater growth, then the fetus is identified as female. If the barley seeds have the greater growth, then the fetus is identified as male.

### CROSS-REFERENCE TO RELATED APPLICATIONS

This nonprovisional application is a continuation of and claims priority to provisional application No. 61/760,873, entitled “Non-Invasive Prenatal Gender Testing Apparatus and Method”, filed Feb. 5, 2013 by the same inventor, which is incorporated herein by reference.

### BACKGROUND OF THE INVENTION

1. Field of the Invention

This invention relates, generally, to gender testing. More particularly, it relates to a non-invasive, organic apparatus and method for determining the gender of an embryo or fetus.

2. Description of the Prior Art

Gender testing that can accurately determine the gender of a fetus typically is invasive and cannot be performed until the fetus is at least 4-5 months old. The most common methodology is a patient undergoing an ultrasound at around 18-22 weeks. However, this method is not conducive for individuals who wish to learn the gender earlier than that time. Determining sex of the fetus by ultrasound is not always successful either, as it heavily depends on the position of the fetus in the womb. Accuracy of ultrasound finding also depends on the age of the baby, the equipment used, the technician, and the cooperation of the baby. Thus, accuracy of the ultrasound is highly subjective, particularly when an ultrasound is conducted earlier than 18 weeks.

An alternative method of determining the gender of a fetus is through amniocentesis, which involved inserting a needle into the uterus to remove amniotic fluid. However, this is done at about 16 weeks and still is not satisfactory for individuals who wish to learn the gender earlier. Furthermore, this type of testing is invasive and comes with risk.

Other tests that can be performed earlier in the pregnancy typically do not provide a satisfactory level of accuracy and are thus rendered useless. Some of these tests offer results at 10 weeks, but the tests urea one-time urine test, thus relying solely on a single urine specimen to determine if the gender is male or female.

Additionally, many myths of gender testing exist, though they are not scientifically validated and are often inaccurate. Examples include carrying position of the fetus, fetal heart beats per minute, carrier sleep position, graceful or clumsy pregnancy, carrier's upset stomach, fetal weight, carrier's soft or dry hands, carrier's food cravings, carrier's acne level upon becoming pregnant, carrier's weight gain in her face, carrier's sugar and spice food cravings, carrier mood changes, pendulum test, hand modeling, advice from a toddler, smells upon consuming garlic, carrier's method of picking up a key, age and year of conception, level of stress at the time of conception, meaning of carrier's dreams, carrier's individual breast sizes, Chinese lunar calendar. Needless to say, these myths are unsubstantiated and are offer no legitimate testing for gender of the fetus.

Another methodology regards the growth of barley and wheat seeds when watered with the carrier's urine. Through translation, ancient Egyptians had produced a test in which a woman who might be pregnant could urinate on wheat or barley seeds over the course of several days. If the wheat seeds sprouted and thrived, the fetus tended to be female, and if the barley seeds sprouted and thrived, the fetus tended to be male. The urine of pregnant women contains elevated levels of estrogen and progesterone; the elevated level of estrogen, in particular, facilitates growth of either seed. Archeological scientists tested this ancient Egyptian methodology and found it to be 70% accurate. However, the exact apparatus and contents of the seeds and soil/dirt/medium were unknown and thus did not permit the methodology to be as effective as it could be.

Accordingly, what is needed is an accurate, objective apparatus and method for testing gender of a fetus at an earlier time in pregnancy. However, in view of the art considered as a whole at the time the present invention was made, it was not obvious to those of ordinary skill how the art could be advanced.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF DESCRIPTION OF THE DRAWINGS

For a filler understanding of the nature and objects of the invention, reference should be made to the following detailed disclosure, taken in connection with the accompanying drawings, in which:

FIGS. 1A-1D depict various components in male and female plants according to an embodiment of the current invention.

FIGS. 2A-2G depict a step-by-step methodology of preparation of seeds in a male plant and a female plant, followed by sprouting of each of the male and female plants.

FIG. 3 is a smartphone screenshot of a software application integrated with an embodiment of the current invention.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

The current invention is a urine-based, at-home gender prediction test that is easy-to-use and thus facilitates identification of the gender of a fetus/baby. Without being pregnant, urinating on seeds or plants typically does not cause seeds to sprout and thrive. However, based on an ancient method created thousands of years ago, two types of seeds are planted, and if a woman is pregnant, hormones in her urine will cause the seeds to grow. The particular seeds that grow stronger and faster determine whether the baby is male or female. Because of the hormonal changes in a woman's system during pregnancy, urinating on particular seeds cause some of those seeds to sprout and thrive. Women who were suspected to be pregnant were advised to urinate on two particular seeds and monitor the growth of the seeds for a period of time. If it was a female child, the one type of seed would grow, and a male child would cause the other type of seed to grow.

In an embodiment, the current invention is an organic gender kit that uses organic wheat seeds and organic barley seeds in a nutrient-rich soil, all contained within clay pots. A prospective mother would supply each soil-seed mixture with her own urine (diluted with water at an approximate 1:1 ratio) over the course of several days with a plurality of applications of said diluted urine. As the seeds begin to sprout, the strength, speed, and height of growth determines whether the fetus is male or female. Whichever seed (wheat or barley) results in the higher strength, speed, and height determines gender of the fetus. Thus, if the wheat seeds have the greater growth, then the fetus is identified as female. If the barley seeds have the greater growth, then the fetus is identified as male.

The methodology has resulted in gender identification with greater than 70% accuracy as early as nine (9) weeks into the pregnancy. This allows a prospective mother to determine the gender of her baby about 50% earlier than the most common sonogram testing utilized at eighteen (18) weeks.

From a scientific standpoint, the baby's gender is not biologically determined until six (6) to seven (7) weeks into pregnancy. At this point, the baby's genitalia begins to develop, and within weeks afterwards, the carrier's urine begins to show traces of male or female hormones produced by the baby. In weeks nine (9) to thirteen (13) of pregnancy, the carrier is producing all of the hormones necessary to determine gender of the baby. The hormones are at full potency to produce an accurate reading, particularly because there are multiple applications of the hormone to the components of the apparatus via the carrier's urine. A sonogram at weeks eighteen (18 to twenty (20) would simply confirm what the current invention has already determined.

Conventional gender tests (e.g., INTELLIGENDER, U.S. Patent App. Pub. No. 2006/0063270, EP 0435940, EP 2257803, U.S. Pat. No. 4,840,914, which are all incorporated herein by reference) are synthetic and/or provide a carrier of a fetus only one chance (i.e., one urine specimen) to accurately identify the gender of the fetus.

Even then, many tests can typically be determined only at a later time period in pregnancy (i.e., 18 weeks or later). Tests that can be performed earlier in pregnancy tend to be minimally accurate, particularly since they allow only a single application (i.e., single urine sample). Utilizing the current invention, the carrier has several opportunities to capture and reinforce her true hormonal balance by permitting multiple urine applications. By being required to provide multiple urine specimens over several days (i.e., hydrating the soil and seeds), a carrier is afforded more than just one opportunity to capture her body's hormonal balance. This ensures a more accurate determination of gender, as the test tracks multiple hormone outputs over a period of time.

Further, the apparatus and methodology of the current invention can be utilized as soon as nine (9) weeks into the pregnancy and still provide accurate results, as the carrier's urine properties are rendered effective at that point for the current invention's methodology. Results become even more accurate as the fetus grows. The best results can typically be obtained at eleven (11) or more weeks into pregnancy.

Occasionally, the male seeds (i.e., barley) and female seeds (i.e., wheat) might both sprout and grow to the same height. This typically occurs when the test is taken very early in the pregnancy and very small amounts, if any, of male gender traits are present in the carrier's urine. In this case, the carrier can count the number of seeds in each pot that have sprouted and compare those numbers. The higher number of seeds that have sprouted typically will indicate the gender of the fetus.

An additional benefit of the current invention is that it can become an educational and enjoyable tool for families and children in particular. Children can learn about the growth of a fetus or baby and can enjoy observing and measuring growth of each set of seeds to determine the gender of a new baby (i.e., sibling, cousin, family member).

System/Apparatus

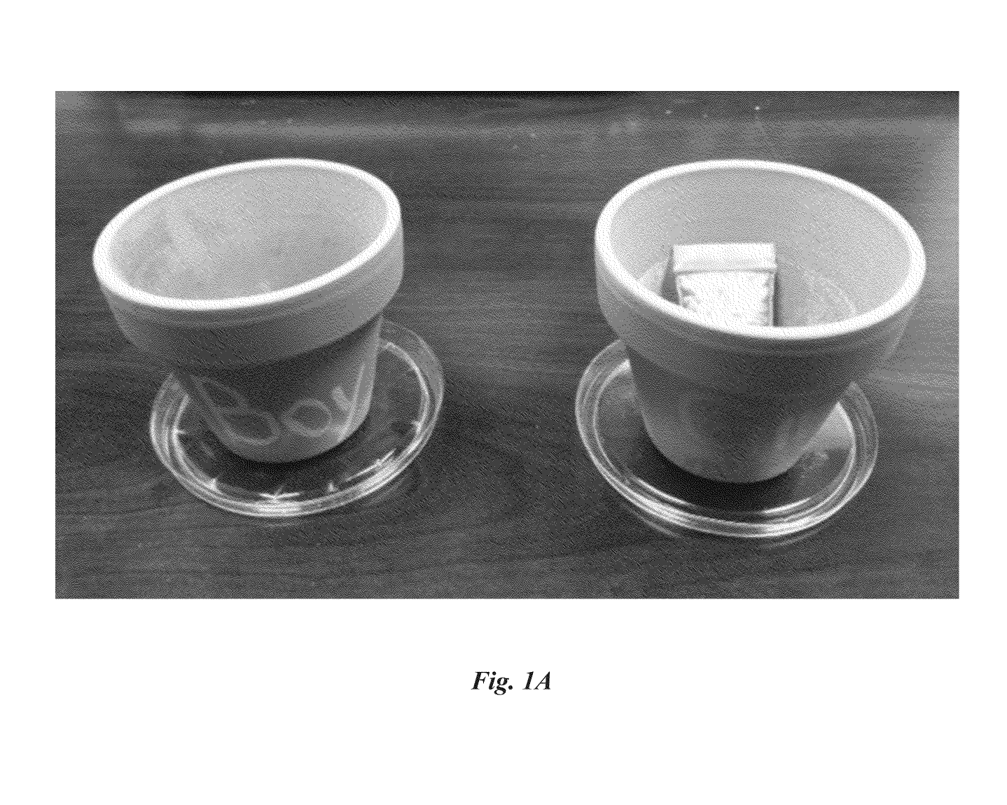

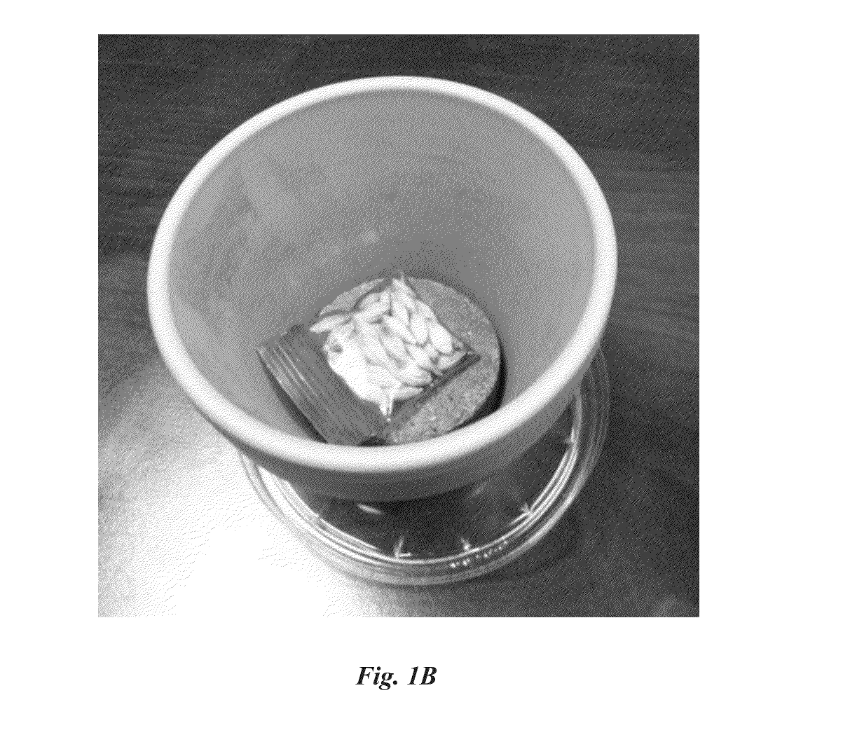

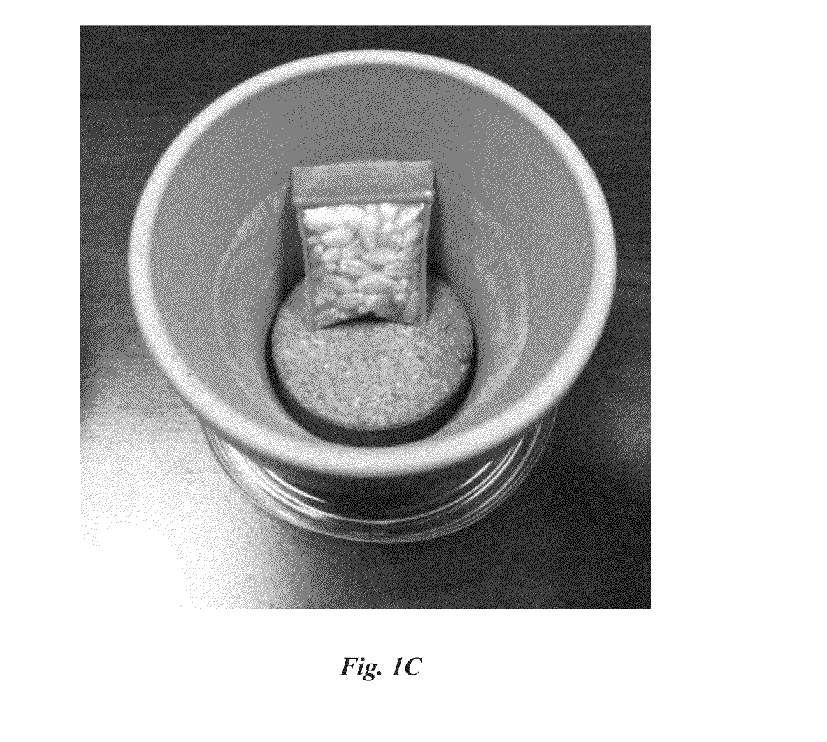

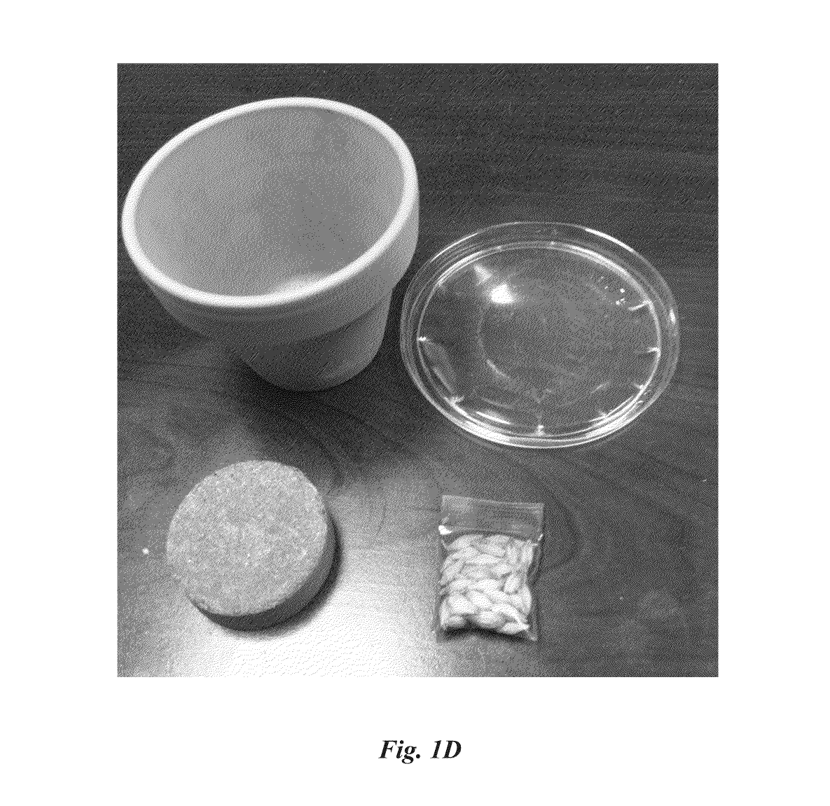

In an embodiment, as seen in FIGS. 1A-1D, the apparatus of the current invention includes two (2) clay pots, each with gender indicator (e.g., label of “boy” or “girl”). The clay pots provide an ideal environment for growth of seeds. The apparatus further includes two (2) seed packs, one (1) with organic wheat seeds and one (1) with organic barley seeds. Each pack as a gender indicator on it (e.g., colored blue for male and pink for female). Both packs also contain the same number of seeds as each other to provide a substantially similar or identical potential for overall growth.

Additionally, the apparatus further comprises two (2) soil pellets, each pellet including about 80% pure coconut husk and about 20% peat pellet. This soil pellet composition is used for prenatal care because of the need for a bland growing medium to prevent any effects on the gender testing. This medium enhances growth of the organic wheat and barley seeds.

The apparatus further includes a specimen cup, two (2) plastic saucers, and one (1) wooden stick. The specimen cup is used for collecting the carrier's urine, diluting the urine, and facilitating the pouring of the diluted urine into the clay pots containing the soil and seeds. The plastic saucers are used as a base for the clay pots to help prevent any spilling or overflow of the urine sample. The wooden stick is used for stirring or mixing the soil pellets and seeds.

Methodology

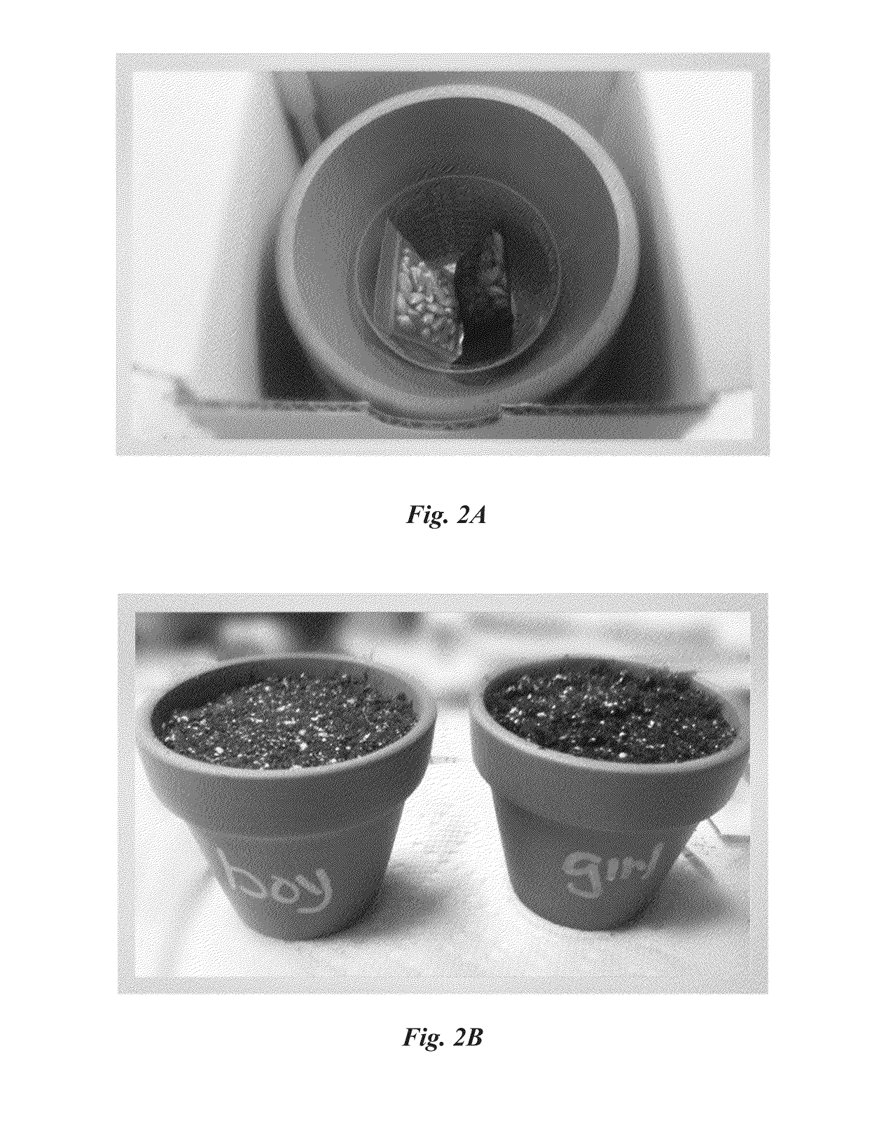

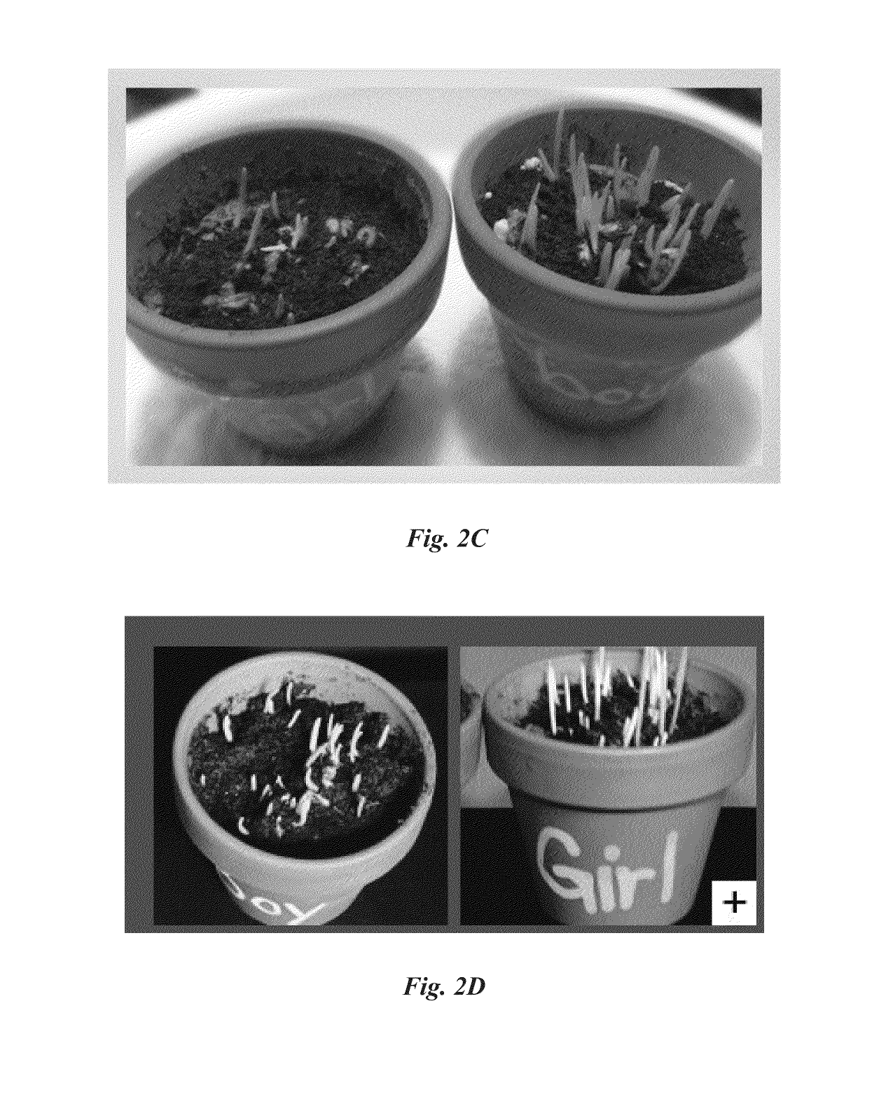

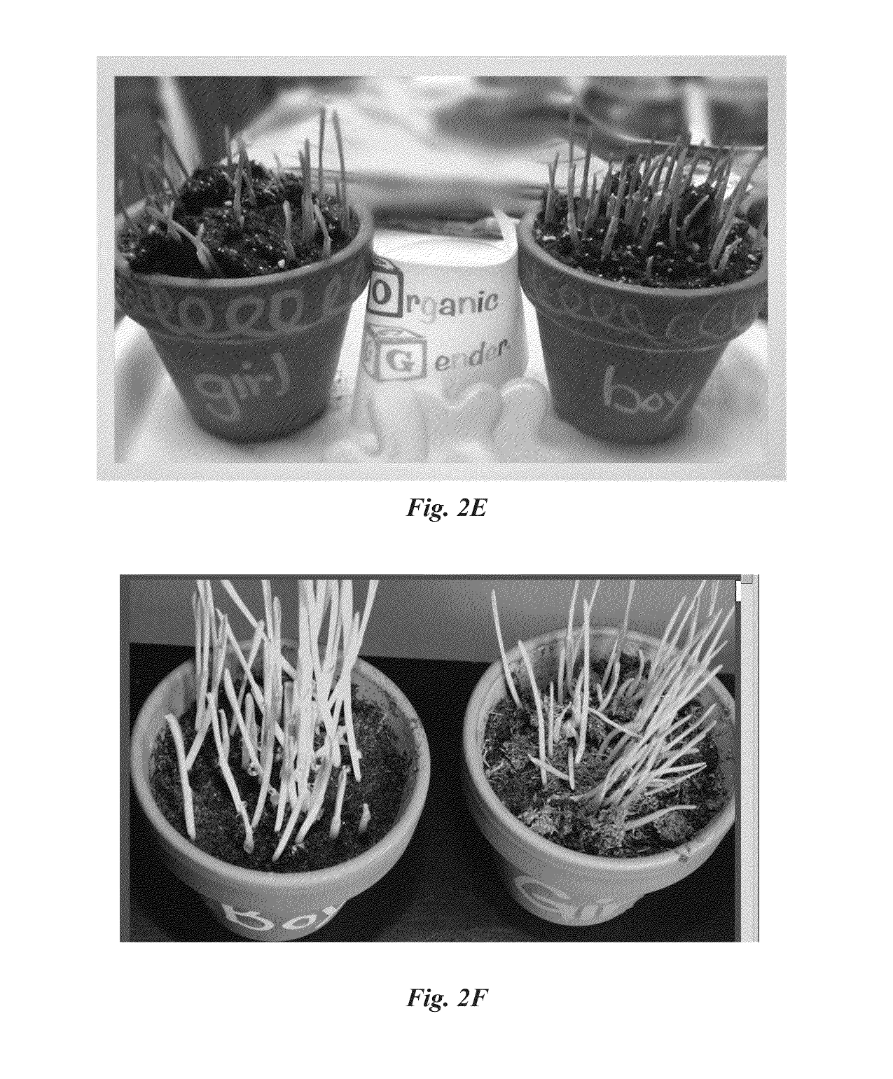

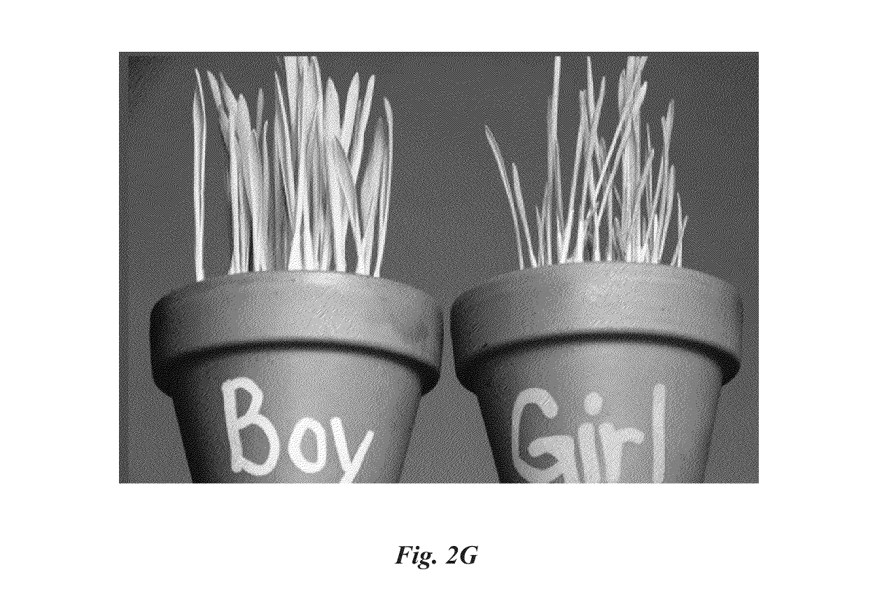

The overall goal is to grow two different sets of seeds combined with the hormones in the urine of the carrier. The more dominant plant determines the gender of fetus. The procedure is depicted in FIGS. 2A-2G.

Utilizing the apparatus of the current invention, each dirt/soil pellet is placed in each clay pot on each plastic sauce. The dirt/soil pellets are rich in nutrients and expand to provide the soil needed in the clay pots. The soil pellets are then hydrated with the carrier's urine via the specimen cup. The carrier's first early morning urine is preferred because it is most concentrated and contains the most desired hormones (alternatively, early morning can be recreated by not using the bathroom for four (4) hours). Prior to hydration, the urine should be diluted with water in about a 1:1 ratio. The initial hydration of the soil pellets needs the highest volume of diluted urine to create the soil. The diluted urine should be poured slowly over the soil pellets and allowed to be absorbed by the pellets. Upon pouring, the pellets should hydrate and expand completely within five (5) to ten (10) minutes to fill the clay pots. The remainder of the soil pellets are mixed with the wooden stick to break apart the soil pellets.

After the pellets are fully hydrated, the top layer of the hydrated pellets (i.e., the peat) is stirred with the wooden stick. More diluted urine can be added if needed. A ditch is then created in the peat in each clay pot. The organic barley seeds are placed into the ditch of the male clay pot, and the organic wheat seeds are placed into the ditch of the female clay pot. All seeds provided should be used, as both sets of seeds contain the same number of seeds as each other to provide a substantially similar or identical potential for overall growth. The peat is then gently manipulated to cover the seeds, so that the seeds are buried just below the surface of the soil (˜⅛^{th} of an inch deep).

ideally, the clay pots with seeds should be placed in a well-lit area, preferably near a window with sunlight exposure. If this is not available, the clay pots can be placed on top of a stove underneath the stove light.

The soil and seeds should be watered about three (3) times over about seven (7) days with the carrier's first early morning urine, diluted with water at a 1:1 ratio. It should be ensured that the manner of preparing and watering the male seeds is the same as the manner of preparing and watering the female seeds. Table 1 depicts an example watering schedule.

TABLE 1
This table explains an example of a watering schedule that can
be used with the current methodology. Results typically would be
seen within five (5) to seven (7) days. The “winning” seeds
may not always be the first seeds to sprout. Rather, the “winning”
seeds might just be the one that grows the strongest and tallest
sprouts over the five (5) to seven (7) day period.
Watering Schedule

| Day Number | Instructions/Notes |
|---|---|
| Day 1 | Plant seeds and hydrate pellets, per previous |
| | instructions |
| Day 2 | Do not water today. |
| Day 3 | Do not water today. |
| Day 4 | Water each clay pot equally by using the |
| | specimen cup with fresh early morning |
| | urine/water, ½ cup on each pot - 1:1 ratio |
| Day 5 | Do not water today. Sprouts may be seen |
| | today. |
| Day 6 | Water each clay pot equally by using the |
| | specimen cup with fresh early morning |
| | urine/water, ½ cup on each pot - 1:1 ratio |
| Day 7 | Observe/Count/Measure sprouts. The |
| | tallest/most sprouts indicates gender of the |
| | fetus. |

Software Application

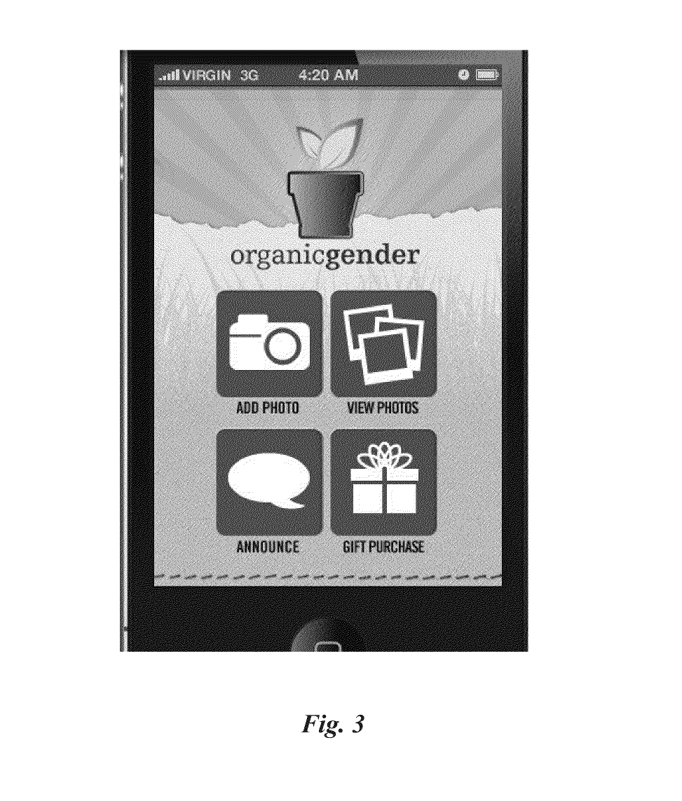

The apparatus and methodology embodiments of the current invention may also be integrated with a software application on a computer system (e.g., smartphone), for example a smartphone application. The software application allows a user to reach out to others with an aft-inclusive socially-connected application. The application does not state the gender of your baby. Rather, it transmits to others observations of the growth of each set of seeds. As seen in FIG. 3, the software application facilitates archiving observations and data for the user and sharing with others, from the beginning of the prediction process through the birth of the baby. As a whole, the current invention includes a system that integrates an apparatus or assembly, methodology, and computer-implemented software application for gender prediction testing, so as to provide the user with an all-inclusive experience crossing into social realms.

Aspects of the present invention are described above with reference to flowchart illustrations and/or block diagrams of methods and/or screenshots, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The advantages set forth above, and those made apparent from the foregoing disclosure, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A method of testing a gender of a fetus prenatally, comprising the steps of:
positioning a first dirt or soil pellet in isolation;
positioning a second dirt or soil pellet in isolation;
hydrating said first pellet with diluted urine of a carrier;
hydrating said second pellet with diluted urine of a carrier;
positioning a barley seed within said first hydrated pellet;
positioning a wheat seed within said second hydrated pellet;
covering said barley seed with peat of said first hydrated pellet;
covering said wheat seed with peat of said second hydrated pellet;
re-hydrating a combination of said first hydrated pellet and said barley seed with diluted urine of said carrier;
re-hydrating a combination of said second hydrated pellet and said wheat seed with a substantially equal amount of diluted urine of said carrier;
administering said re-hydration steps about three (3) times per day for about seven (7) days;
comparing plant growth between said barley seed and said wheat seed, wherein enhanced plant growth of said wheat seed indicates a female gender of said fetus and wherein enhanced plant growth of said barley seed indicates a male gender of said fetus.
